(19) United States
(12) Patent Application Publication
(10) Pub. No.: US 2023/0191095 A1
(43) Pub. Date: Jun. 22, 2023

(54) Inline Pressure and Temperature Sensor for Cerebral Shunts

(71) Applicant: Cognos Therapeutics Inc., Inglewood, CA (US)

(72) Inventors: Thomas C. Chen, La Canada, CA (US); Josh Shachar, Santa Monica, CA (US); Thomas Lobl, Valencia, CA (US); Winston Wu, Alhambra, CA (US)

(73) Assignee: Cognos Therapeutics Inc., Inglewood, CA (US)

(21) Appl. No.: 17/560,058

(22) Filed: Dec. 22, 2021

(51) Int. Cl. A61M 27/00; A61B 5/03

(52) U.S. Cl. CPC A61M 27/006; A61B 5/032; A61B 2560/0223; A61M 2205/3334; A61M 2205/3553; A61M 2202/0464; A61B 2562/0247; A61B 2562/0271; A61B 2562/0238; A61M 2205/3584

(57) ABSTRACT

The invention includes a ventriculo-peritoneal shunt and a method of operating it. The shunt includes: a ventricular catheter for transferring CSF from the ventricle of a brain of a patient; a pressure sensor communicated to the ventricular catheter to measure the pressure of the CSF as delivered to the pressure sensor; a temperature sensor communicated to the ventricular catheter to measure the temperature of the CSF as delivered to the temperature sensor; a wireless data transmitter to transmit the measured pressure and temperature to an attending physician; a nonprogrammable reporting valve or programmable valve communicated to the pressure sensor and temperature sensor to regulate flow of the CSF; and peritoneal tubing communicated to the programmable valve for delivering CSF to the peritoneal cavity.

### BACKGROUND

### Field of the Technology

The invention relates to methods and apparatus for brain shunts for the treatment of hydrocephalus.

### Description of the Prior Art

Hydrocephalus is an abnormal accumulation of CSF fluid in the brain which can lead to brain stem compression which is life threatening. Hydrocephalus can occur in children as well as in adults and be caused by congenital causes or from accidents and head trauma. Hydrocephalus shunts (HS) are normally required for the entire life of the patient but have many issues and current products are less than ideal. Nearly 30% fail within one year of the initial surgery. Virtually 100% fail within ten years due to clogging and infection. There are two main types of hydrocephalus: obstructive which is caused by a blockage in drainage and communicating where there is a problem in reabsorption of cerebral spinal fluid (CSF).

A shunt is a device that is used to divert flow from one part of the body to another. For the central nervous system, they are usually called ventriculo- (diversion from ventricle of the brain), lumbo- (diversion from lumbar thecal sac), cysto- (diversion from a cyst), or lepto-meningeal (diversion from subarachnoid space). These shunts then deliver their fluid contents into another part of the body i.e. peritoneal (into the peritoneum), atrial (into the atrium), or pleural (into the pleura of the lung). The most common types of shunts are the ventriculo-peritoneal shunt, the ventriculo-atrial shunt, the ventriculo-pleural shunt, the lumbo-peritoneal shunt, and the lumbo-pleural shunt.

The most common condition requiring a shunt is called hydrocephalus, a condition in which there is a buildup of cerebrospinal fluid (CSF) resulting in increased intracranial pressure. Hydrocephalus can result in significant morbidity or even death if not treated in a timely manner. The shunt treats hydrocephalus in an effective and timely manner and has saved many patients' lives.

Currently, a shunt is made up of three main components: a ventricular catheter, a valve, and a tubing that can be placed into the peritoneum, pleura, or atrium. Each of these components are made of a biocompatible plastic such as silicone rubber. The ventricular catheter is used for placement into the ventricle of the brain. It is connected in tandem to a valve which has a pressure release mechanism, like an elastomeric duckbill valve, that releases CSF when it is over a predetermined pressure. Some valves have now improved to the point that they can be mechanically adjusted using an external magnet to open and close the valve at the patient's option without any reading or sensing of pressure by which CSF is diverted. Such valves are not MRI compatible. The downstream tubing has its input connected to the valve, and its output is placed into the peritoneum, atrium, or pleura, where the CSF can be reabsorbed. Depending on body size our body has approximately 150 cc of CSF, which is turned over three times a day. So, if there is a problem with the drainage or reabsorption, hydrocephalus will develop. If the shunt is not working properly, the patient will become symptomatic with headaches, dizziness, blurred vision and other issues.

The two main complications of shunts currently are obstruction and infection. Most shunts fail because of obstruction (in which the intraventricular catheter becomes plugged, or the peritoneal tubing becomes plugged). When a shunt becomes obstructed, the patient develops signs and symptoms of hydrocephalus. In a minority of cases, a shunt can also become infected, it then would have to be removed, and the patient treated with systemic antibiotics.

The global hydrocephalus shunts market size was valued at $291.00 million in 2019, and is projected to reach $322.01 million by 2027, registering a CAGR of 2.8% from 2020 to 2027. Congenital hydrocephalus is considered a rare disease which affects less than 200K people in the US. Incidence is about 1/105K (2,580/yr) in the US and 6.46/10K births in the UK. Overall prevalence comes to about 0.5%. Shunt failure accounts for 50% of all pediatric neurosurgeries and about $3B a year in health care expenditures. In US about 125K shunt procedures are done each year at a cost of about $100M; in UK/Ireland 3.5-4K procedures/yr. About 3-9% of severe head injuries develop communicative hydrocephalus.

Hydrocephalus can be a fatal if not managed properly. HS fail remarkably often due to obstruction, over-drainage and changes in regulating valve performance. Under or over-drainage leads to pain, nausea, fatigue, abnormal gait, incontinence, poor cognition and many more symptoms. These symptoms send the patient to hospital for MRI or CT scans or putting a pressure sensor via a needle through the skin and into the shunt catheter. Current hydrocephalus shunts do not provide real-time brain pressure status information or allow for convenient adjustment to intracranial pressure. In other words, patients do not know if the HS has failed, and they now have high (or low) brain pressure. Patients have to go to the hospital emergency room (for CT or MRI) to learn if the reason they feel bad is because of high brain pressure. It is a cause of many “unnecessary” ER visits and expensive CT/MRIs.

The consequences of hydrocephalus can be life threatening at worst and at best brain damaging, if not managed well actively. This requires the patient with hydrocephalus to be ever vigilant when a headache or other symptoms occur. The patient and even the doctor does not have a good tool to monitor changes in brain pressure be they be non-consequential or dangerous. For the patient's ease of mind and in today's modern life there is a need for continuous pressure brain monitoring for the hydrocephalus patient and indirectly for the doctor.

What is needed is an apparatus and method of operating the apparatus which overcomes each of the above problems.

### BRIEF SUMMARY

The illustrated embodiments of the invention address the two main complications that may occur from a shunt-obstruction and infection. An in-line sensor is employed that is able to detect pressure and temperature. The inline sensor is placed as an extension of the current shunt. It is distal to the ventricular catheter, proximal to the valve and tubing. If a shunt is obstructed proximally, it will demonstrate decreased pressure as the obstruction is at the tip of the catheter and CSF is not flowing in. If the obstruction is at the level of the valve or peritoneal tubing, it will demonstrate increased pressure, as the CSF is now held up at the valve or distal to it. If a shunt is infected, the CSF temperature will be elevated. These readings can be transmitted wirelessly to the physician, who will then be able to make a diagnosis of possible shunt malfunction or infection when the patient presents to the office or hospital emergency room with these possible complications. The patient will also simultaneously receive an alarm from the smart shunt system on his phone app suggesting the patient should notify the doctor and go to the emergency room to be checked out.

Additionally, low CSF pressure is a serious medical issue too. In this case the valve has been open too long and released too much CSF. When this happens the shunt pressure and temperature system will also notify the doctor and the patient about the low pressure and to go to the emergency room. This situation is when the sensors do not operate the valve to open or close it. This occurs when the valve is not controlled by the electronics in a closed loop manner. Note that the illustrated embodiments include systems that just measure pressure and temperature and notify the doctor and/or patient and embodiments where the smart shunt system regulates the valve as a closed loop system.

To address this, the illustrated embodiments include an implanted, non-drifting pressure sensor that would be sensltive enough to measure CSF pressure real-time. Two technologies are available that have the capabilities to meet this need. One is an electrically powered piezo-based strain gage. The other is optical based. Optical pressure measurements are based on sending light to a pressure-senslng, mirrored diaphragm and measuring the reflected light phase changes. This requires a light source and dependng on the design, optionaHy, no electronics.

The illustrated embodiments of the invention thus include a ventricular shunt having a controllable valve to regulate flow of cerebral spinal fluid (CSF) from the brain of a patient for treatment of hydrocephalus comprising: a ventricular catheter for transferring CSF from a brain ventricle of a patient; a pressure and/or temperature sensor communicated with the CSF to measure pressure and/or temperature respectively of the CSF; a wireless data transmitter to transmit the measured pressure and/or temperature to an attending physician and/or patient; and a drainage catheter communicated to the controllable valve for draining the CSF from the brain. The ventricular catheter has a catheter tip, the valve has an inlet, and the pressure sensor is disposed at or near the ventricular catheter tip and/or at or near the inlet of the valve, so that a high pressure measurement by the pressure sensor at either the catheter tip and/or at or near the inlet of the valve less than a predetermined magnitude is reported by the wireless data transmitter as a clogged ventricular catheter to the attending physician and/or patient.

For example, the predetermined magnitude is in the range of 5-15 mmHg is considered normal pressure in a supine adult. Any pressure greater than 20-30 mm Hg is considered mild brain hypertension and pressures of 40 mm Hg or higher are considered life threatening. A pressure below 0 mm Hg (depending on the source and/or 0 to −15 mm Hg) is considered low pressure. Low pressure is a frequent problem with shunts causing over drainage with open valves. A sensor managed active valve that operates in a closed loop is a significant improvement. It will not just open to relieve high pressure but also prevent over drainage.

In one embodiment the pressure sensor includes multiple pressure sensors, selected ones of the multiple pressure sensors disposed at or near the ventricular catheter tip and/or at or near the inlet and/or outlet of the valve, so that a high pressure measurement by selected ones of the multiple pressure sensors at or near the ventricular catheter tip and/or at or near the inlet of the valve greater than a predetermined magnitude is reported by the wireless data transmitter as a clogged valve to the attending physician and/or patient, or a high pressure measurement by selected ones of the multiple pressure sensors at or near the outlet of the valve greater than a predetermined magnitude is reported by the wireless data transmitter as a clogged drainage catheter to the attending physician and/or patient. For example, predetermined magnitude is in the range of ≥20 mmHg.

The brain, as a high energy consuming organ, requires a stable temperature to operate normally and is sensitive to temperature changes. The CSF plays an important role in maintaining this temperature. A temperature higher or lower than normal would indicate a diseased condition or an infection. Temperature measurements outside of normal would be an important indicator of disease for patients. Normal temperatures for men are 36.7+/−0.7° C. and for women are 37.5+/−1.3° C. or an average of 36.9+/−0.4° C. and changes during the day depending on activity, resting or sleep. A high temperature measurement by the temperature sensor greater than a predetermined magnitude is reported by the wireless data transmitter as an infection in the patient. For example, the predetermined magnitude is in the range of ≥2° C. from the average. In addition, temperature measurements are needed to accurately calculate pressures measured with a piezo strain gauge.

In a first embodiment the wireless data transmitter includes an external RFID scanner and an implantable body. The implantable body includes: an RFID transceiver wirelessly communicated to the RFID scanner; a battery; a battery charger circuit coupled to the battery and to the RFID transceiver; a power reducing circuit; and a programmable microcontroller coupled to the RFID transceiver, power reducing circuit and to the pressure sensor and/or temperature sensor, where the microcontroller is programmed to collect, store and/or transmit data from the pressure sensor and/or temperature sensor and to control operation of the RFID transceiver and the pressure sensor and/or temperature sensor, whereby measured pressure and/or temperature of the CSF relating to the operational status of the shunt is wirelessly reported to a physician and/or patient for interventional response.

In a second embodiment, the wireless data transmitter includes an external RFID scanner and an implantable body. The implantable body includes: an RFID transceiver wirelessly communicated to the RFID scanner; a battery; a battery charger circuit coupled to the battery and to the RFID transceiver; a power reducing circuit; and a programmable microcontroller coupled to the RFID transceiver, power reducing circuit, to the pressure sensor and/or temperature sensor and to the controllable valve, where the microcontroller is programmed to collect, store and/or transmit data from the pressure sensor and/or temperature sensor and to control operation of the RFID transceiver, the pressure sensor and/or temperature sensor, and the controllable valve in either a closed or open loop configuration, whereby measured pressure and/or temperature of the CSF relating to the operational status of the shunt is wirelessly reported to a physician and/or patient when in the open loop configuration for interventional response, or is communicated to the microcontroller for control of the valve when in the closed loop configuration and simultaneously to the patient and doctor for confirmation of its “automatic” actions and the device data log.

. In a third embodiment the wireless data transmitter includes an external RFID scanner and an implantable body. The implantable body includes: an antenna wirelessly communicated to the RFID scanner; a low energy Bluetooth circuit coupled to the antenna; a power reducing circuit; a capacitor for storage of power coupled to the power reducing circuit; and a programmable microcontroller coupled to the Bluetooth circuit, power reducing circuit, and to the pressure sensor and/or temperature sensor, where the microcontroller is programmed to collect, store and/or transmit data from the pressure sensor and/or temperature sensor and to control operation of the Bluetooth circuit, the pressure sensor and/or temperature sensor, where power is transmitted from the RFID scanner through the antenna, Bluetooth circuit, microcontroller, and power reducing circuit to the capacitor, and whereby measured pressure and/or temperature of the CSF relating to the operational status of the shunt is wirelessly reported to a physician and/or patient for interventional response.

The illustrated embodiments of the invention also extend to a method of using a ventricular shunt having a controllable valve to regulate flow of cerebral spinal fluid (CSF) from the brain of a patient for treatment of hydrocephalus including the steps of: delivering CSF from the ventricle of a brain of a patient through a ventricular catheter; measuring pressure and/or temperature of the CSF using a pressure sensor and/or temperature sensor respectively communicated to the ventricular catheter as delivered to the pressure and/or temperature sensor; transmitting the measured pressure and/or temperature to a physician and/or patient using a wireless data transmitter; regulating flow of the CSF using a controllable valve communicated to the pressure sensor and/or temperature sensor; and transferring the CSF through a drainage catheter communicated to the controllable valve to a body situs for CSF absorption.

The step of measuring pressure of the CSF determines if a low-pressure measurement by the pressure sensor is less than a predetermined magnitude and reporting the determined low pressure using the wireless data transmitter to the attending physician and/or patient as a clogged ventricular catheter.

The step of measuring pressure of the CSF determines if a high-pressure measurement by the pressure sensor is greater than a predetermined magnitude and reporting the determined high pressure to the attending physician using the wireless data transmitter to the attending physician as a clogged programmable valve or clogged peritoneal tubing.

The step of measuring the temperature determines if a high temperature measurement by the temperature sensor is greater than a predetermined magnitude and reporting the high temperature to the physician and/or patient using the wireless data transmitter as an infection in the patient.

In one embodiment the step if regulating flow of the CSF using a controllable valve communicated to the pressure sensor and/or temperature sensor is performed by physician and/or patient intervention in an open loop control of the valve.

In another embodiment the step of regulating flow of the CSF using a controllable valve communicated to the pressure sensor and/or temperature sensor is controlled by a programmable microcontroller coupled to the valve and to the pressure and/or temperature sensor for closed loop control of the valve.

In the embodiment where ventricular catheter has a catheter tip, and the valve has an inlet and outlet, and where the pressure and/or temperature sensor include a plurality of pressure sensors communicated to the CSF at or near the catheter tip, the inlet or the outlet of the valve, the step of measuring pressure and/or temperature of the CSF is performed at or near the catheter tip, the inlet or the outlet of the valve, and where regulating flow of the CSF using a controllable valve communicated to the pressure sensor and/or temperature sensor is performed based on an interpreted operational status of the shunt based on pressure and/or temperature measurements at or near the catheter tip, and/or the inlet or the outlet of the valve.

Still further, the illustrated embodiments include an optical sensor system for measuring CSF pressure in a brain of a patient including: an implanted optical sensor for receiving light and returning phase shifted light indicative of the CSF pressure in the brain; source of light for generating light communicated to the optical sensor; an exterior reader for reading the CSF pressure in the brain as indicated in the returned phase shifted light from the optical sensor; and means for communicating the CSF pressure in the brain as indicated in the returned phase shifted light from the optical sensor to the reader or for communicating the returned phase shifted light from the optical sensor to the reader.

In one embodiment the source of light is exterior to the patient and the means for communicating the returned phase shifted light from the optical sensor to the reader includes an implanted optical terminus and an optical cable communicating the implanted optical sensor with the implanted terminus, the exterior reader optically coupling with the implanted terminus.

In another embodiment the source of light is implanted in the patient and the means for communicating the CSF pressure in the brain as indicated in the returned phase shifted light from the optical sensor to the reader includes an implanted wireless communication circuit electromagnetically communicating with the exterior reader.

In one embodiment the source of light and means for communicating the CSF pressure are included within a single implanted housing. In another embodiment the source of light and means for communicating the CSF pressure are included within a plurality of intercommunicated implanted housings.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a diagram showing a ventriculo-peritoneal shunt as used in the prior art.

FIG. 2 is a diagram showing a ventriculo-peritoneal shunt according to the illustrated embodiments of the invention.

FIG. 3 is a diagram showing a side view of the pressure and/or temperature sensor as used in FIG. 2.

FIG. 4 is a block diagram an active battery-powered sensor system to measure pressure and temperature and report this data back to an external controller.

FIG. 5 is a block diagram of an active battery-powered sensor system that is connected to a valve that can be opened or closed to modulate brain pressure based on the data collected by the sensor and interpreted by the microcontroller.

FIG. 6 is a block diagram of a fully implanted passive sensor system.

FIG. 7 is a diagram showing from left to right depictions of a conventional hydrocephalus shunt, a shunt with an intraventricular sensor on the catheter tip, and a three-sensor shunt.

FIG. 8 is a diagram that illustrates a passive sensor system according to one embodiment.

FIG. 9 is a side cross-sectional view of the optical pressure sensor with a single optical cable as used in the illustrated embodiments.

FIG. 10 is a side cross-sectional view of the optical pressure sensor with a dual optical cable as used in the illustrated embodiments.

FIG. 11 is a side cross-sectional view of a dual optical cable assembly included in a single sheath.

FIG. 12 is a transparent perspective view of a single cable terminus.

FIG. 12a is a side cross-sectional view of the terminus of FIG. 12.

FIG. 13 is a transparent perspective view of a dual cable terminus.

FIG. 13a is a side cross-sectional view of the terminus of FIG. 13.

FIG. 14 is a block diagram of a first embodiment of an active pressure system, wherein the entire implanted system is included in a single housing.

FIG. 15 is a block diagram of a second embodiment of an active pressure system, wherein the implanted system is divided between two housings.

FIG. 16 is a block diagram of a first embodiment of the reader used with a passive pressure sensor.

FIG. 17 is a block diagram of a second embodiment of the reader used with active pressure sensors.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

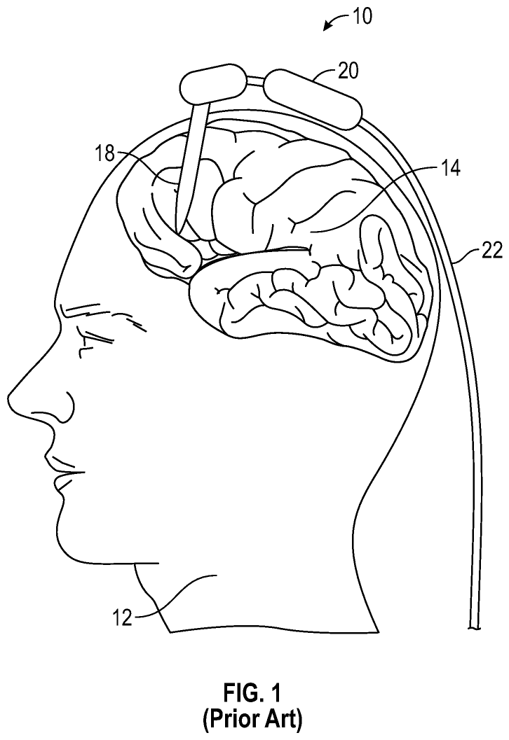

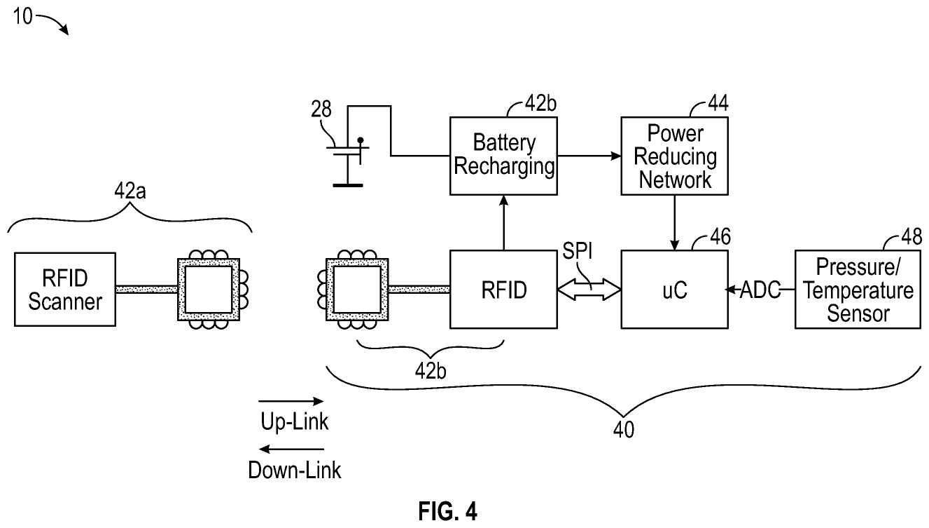

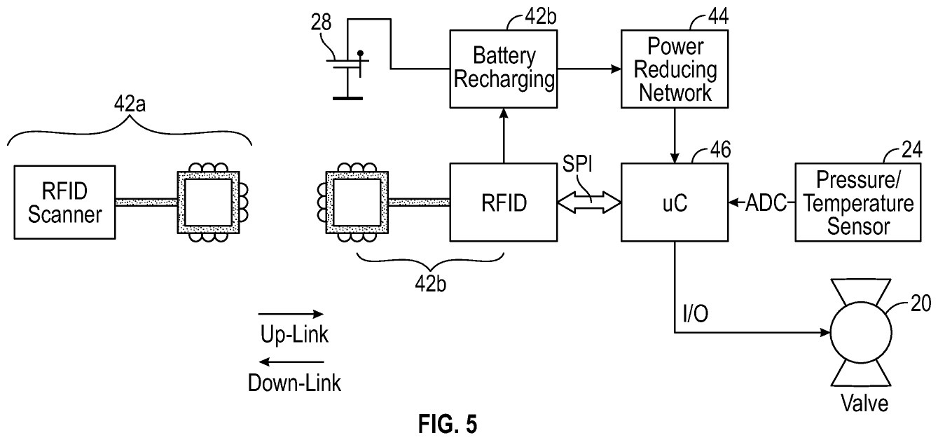

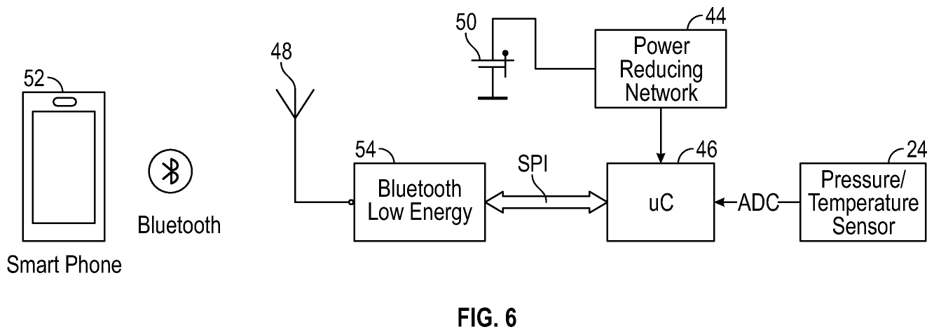

The illustrated embodiments of the invention include three preferred embodiments for a fully implanted hydrocephalus pressure sensing system as described below in connection with FIGS. 4-6, after briefly addressing prior art shunt technology and the general approach of the smart shunt of the illustrated embodiments in connection with the FIGS. 1-3. The first embodiment of FIG. 4 is an active battery-powered sensor system to measure pressure and temperature and report this data back to an external controller. The second embodiment of FIG. 5 is an active battery-powered sensor system that is connected to a valve that can be opened or closed to modulate brain pressure based on the data collected by the sensor and interpreted by the microcontroller and report this data back to an external controller. The third embodiment of FIG. 6 is a fully implanted passive sensor system. The third embodiment collects data when connected to an external RFID controller that provides power to the sensor to collect immediate data. This third embodiment does not collect or store data between queries by the external controller.

It is to be understood that all of these embodiments include proprietary software to operate the medical device(s) and the appropriate graphical user interfaces appropriate for the patient, the doctor and for storage in EMRs (electronic medical records), the cloud, and for big data and Al analyses.

FIG.1 is a diagram of a ventriculo-peritoneal shunt 10 as used in the prior art. Patient 12 has a distal end of a ventricular catheter 16 implanted into the ventricle 18 of his or her brain 14. The proximal end of catheter 16 is communicated to a valve 20 described above, which is preferably located chest area. The outlet of valve 20 is communicated to a peritoneal tube 22, whose distal end empties into the peritoneal cavity of the abdomen of patient 12. Thus FIG. 1 illustrates a conventional shunt 10 with a ventricular catheter 16 going into the ventricle of the brain 14, a passive valve 20 that can be opened or closed when the CSF pressure at which CSF too high or low to modulate the rate of release into the peritoneum or other selected site and a peritoneal tubing 22 conducting CSF into the peritoneum where it is reabsorbed. This valve 20 can be operated closed loop using data from the pressure sensor or open loop by notifying the patient and doctor that the valve needs to be adjusted using a separate device for manually operating the valve.

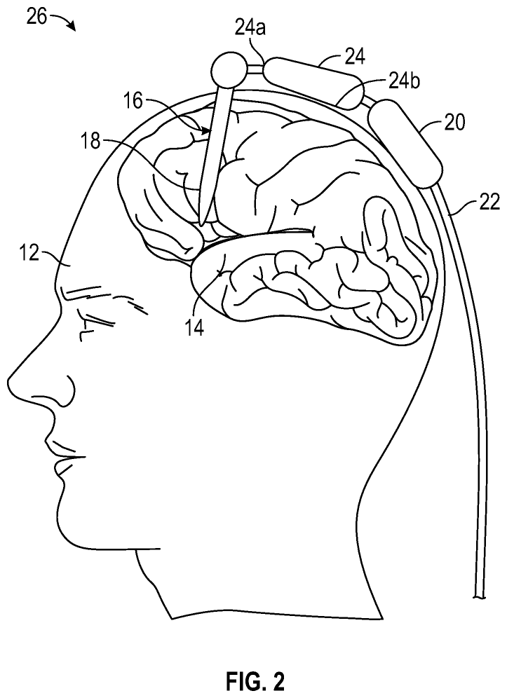

FIG. 2 is a diagram of the smart shunt 26 of the illustrated embodiments with an in-line sensor 24 for pressure and temperature monitoring, optionally located within the same housing. The temperature sensor may be optional as it measures infection, which is usually a rare event. However, temperature is needed to accurately calibrate the pressure sensor. Body temperature is relatively constant so it can be assumed if the temperature sensor is absent. In FIG. 2 the sensor 24 is whown outside the skull, but it can also be placed in the ventricle or in the chest area. The new in-line sensor 24 is placed distal to the ventricular catheter 16 and proximal to the valve 20. In FIG. 2 The peritoneal tubing 22 is distal to the valve 20. The inline sensor 24, which includes a pressure sensor 24a and temperature sensor 24b. The pressure sensor 24a monitors the intracranial pressure of patient 12. The temperature sensor 24b monitors monitors temperature and notes any change in temperature. Intern lcranial pressure (ICP) and temperature are transmitted wirelessly to the attending physician and to an app on the patient's phone or other receiver if it is not convenient to receive this data on a phone.

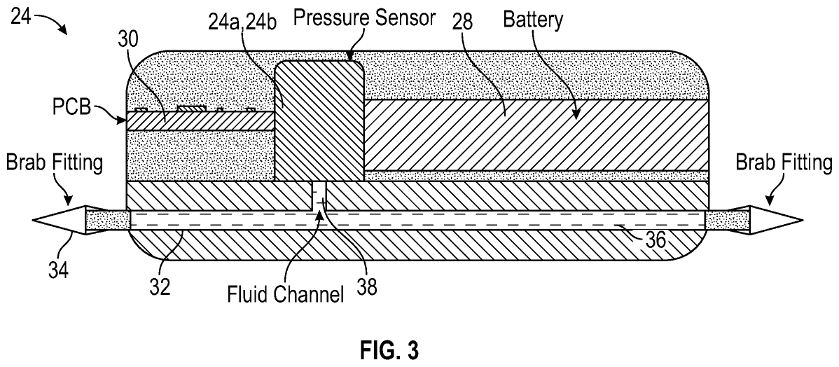

FIG. 3 is a block diagram illustrating the internal layout of inline sensor 24. The pressure and temperature sensors 24a and 24b respectively are an element in an MRI-compatible, nonprogrammable or programmable valve with eight different pressure settinas used in the situation of FIG. 1, including a ‘virtual off’ setting, for the treatment of hydrocephalus as currently made under the brand Codman Certas by

Integra Life Sciences of Plainsboro, N.J. Medtronics of Minneapolis, Minn., also manufactures a plurality of shunts used in the context of FIG. 1 under the brands, Strata Valves, Delta Valves, CSF-Flow Control Valves and LP Shunts, all of which include some type of hydrocephalus pressure sensor. The same or similar type of sensor can be modified and employed in the context of FIG. 2.

The inline sensor 24 has two male and-female connectors 34. The male connector 34 is included in the in-line sensor 24 and connects to a conventional female lumen of the ventricular catheter 16. The posterior part 36 of the inline sensor 24 connects to the programmable valve 20, A fluid channel 32 is provided through sensor 24 from the proximal connection 34 to the distal connection 36. A via (fluid channel) 38 communicates channel 32 to sensors 24a and 24b to provide pressure and temperature measurements respectively. The operation of sensor 24 is controlled by digital circuitry mounted on printed circuit board (PCB) 30. Programmability of wireless communication as well as measurement functions are provided by the circuitry of PCB 30. The circuitry and sensors 24a and 24b are powered by an inductively rechargeable battery 28.

### First Embodiment

FIG. 4 is a block diagram for a fully implanted pressure and temperature sensor system 10. The implanted system 10 is battery powered. The battery 28 can be recharged as needed through the combination of an external and internal an antenna and radio frequency identification (RFID) system 42a and 42b respectively or optionally any other system designed for recharging implanted batteries that is powered from an outside the body to recharge an internal battery as needed. RFID systems 42a and 42b essentially serve as a keyed input and output interface. The battery 28 adjusts the power through a power reducing network 44 designed for providing power to the microcontroller 46 (μC) which manages all the internal functions of the system 10. The μC 46 also manages the pressure and temperature sensors 24 and stores data from the sensors 24 in the memory of μC 46 until downloaded to the RFID scanner 42a and then to physician and patient controllers (not shown). The physician and patient controllers arc communicate with valve 20 to open or close valve 20 according to the decision of the physician, patient or programmed routines resident in the physician and patient controllers. In the case of a nonprogrammable valve, the system just reports a problem to the patient and/or doctor. After the data is downloaded, the μC 46 deletes the data from its memory to make room for more data. The memory will hold preferably at least 6 months of data.

The μC 46 also controls all the necessary functions of the system 10 so it may operate as a fully autonomous system run from an internal software program that is modifiable by the doctor and or patient as needed, including but not limited to battery function and recharging, sensor operation and data collection and storage, and communication with the outside world through the implanted antenna 42b. The sensor module 24 is communicated to the cerebral spinal fluid (CSF) and can measure CSF pressure and/or CSF temperature. If the pressure is too high or low or if the temperature is too high or low, indicating an infection or other issue, the μC 46 sends an alarm to the patient and, if programmed this way, immediately to the doctor simultaneously or at a later time through the RFID controller 42b. The μC 46 also controls the data uplinks and downlinks with external devices through a radio channel, the RFID or other nearfield communications system and through a smart phone 52, or optionally another receiving/sending device, communicated with either the physician portal and or controller (not shown). The external RFID scanner 42a and the attached antenna 42a may optionally also be a radio link to the implanted device via an antenna from the physician controller, a computer system or a patient pressure/temperature monitoring device, e.g. smart phone 52, that calculates and displays the data via a proprietary algorithm on an smart phone 52 or other convenient device or computer that is capable of receiving the data and alerts should there be a problem with the brain pressure or temperature. The data will also optionally be formatted for storage in electronic medical records and stored in the cloud for big data analysis and Al studies.

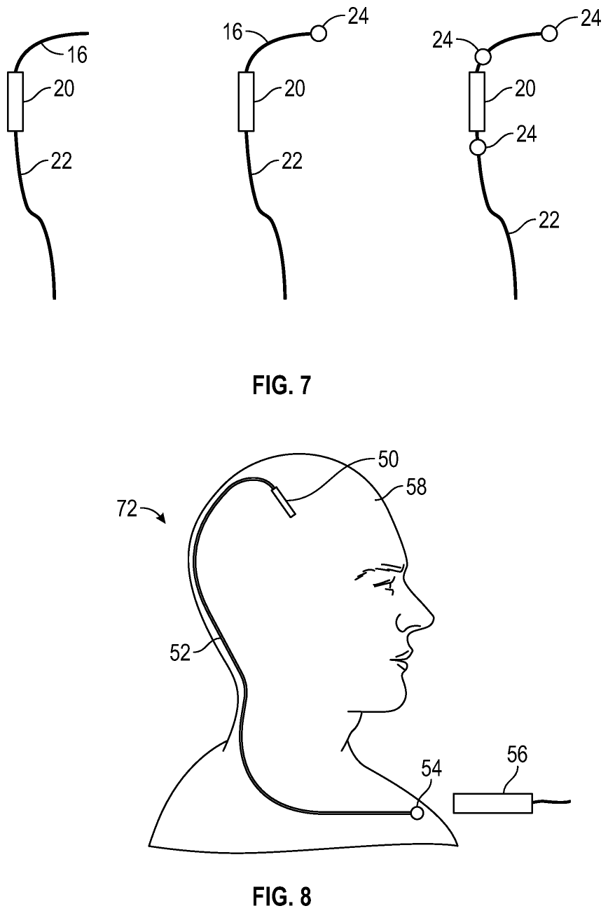

It should be noted that the sensors 24 may be incorporated into one of lumens in the shunt's catheter 16 or optionally be implanted separately from the shunt's catheters 16. Although this example illustrates only one sensor 24 there may be several more with 1-3 sensors being the preferred number of sensors. In the case of three sensors as shown in the right-most depiction in FIG. 7, one sensor is exposed directly to the ventricle CSF, an optional second sensor is exposed to the lumen of catheter 16 immediately before the shunt valve and a third sensor is placed in the downstream drainage catheter or tubing 22 before the exit to the peritoneum or other exit location. Each of the sensors 24 is wired directly to the implant body 40 in which μC 46 is located. The wires from the sensors 24 can be carried to the implant body 40 through a lumen in a multi-lumen catheter 16 where one lumen carries CSF through, and one or more lumen carry the necessary wires to operate the sensors 24 and connect them to the implant body 40. Optionally and less preferred, the wires are not be part of the catheter communicating with brain and peritoneum.

### Second Embodiment

The second embodiment shown in FIG. 5 is similar to the first embodiment except that it additionally has the ability to control a shunt in-line valve 20. Valve 20 is connected to a catheter 16 whose tip is upstream in the brain ventricles and is able to deliver CSF from the brain through valve 20 to downstream tubing 22 that is connected to the other side of the valve 20 and delivers the brain CSF to the peritoneum or elsewhere as appropriate when the valve 20 is open. In this way the system 10 may be operated as an opened or closed loop system. In the open loop mode, the valve 20 can be opened and closed via an externa signal from outside the body (such as a magnet) or electronic signal to the valve's electronic system or the μC 46 of the implanted device. Alternatively, the valve 20 may be opened or closed when the sensors 24 identify the brain pressure is too high or too low. In this latter case, valve 20 is operated in a closed loop mode. In the closed loop mode, the μC 46 analyses data from the sensors 24 and according to preset values makes a decision to open or close the valve 20 by sending a signal and the necessary power to the valve's electronics to operate the opening or closing mechanism of the valve 20. The valve 20 will also have the analytical capability via its internal programming and appropriate sensor 24 to inform/confirm to the μC 46 that the appropriate actions of the shunt valve 20 have been taken. Most preferred in the closed loop operational mode, the valve 20 should be physically close to the implant body 40. In this case the preferred configuration is to have the shunt valve 20 and the pressure/temperature sensor 24 inline and open or in contact with the catheter CSF. In this case the valve 20 can be incorporated into the body of the sensor 24 for efficient management of the valve 20 by the sensor electronics. However, in this case if there are other sensors in line on the system, then they are not connected to the valve 20 and only report the pressure and temperature and don't have authority to open or close the valve 20. The microcontroller 46 will make the decisions based on one or more of the sensor's reporting. If the sensors 24 give conflicting information, then the microcontroller 46 makes the decision or sends the information to the doctor for the doctor to resolve. This could happen if one part of the tube is blocked but the other is not. Alternatively, if one sensor is blocked or non-functional and the others are not.

Alternatively, the shunt valve 20 can be incorporated and internalized into the body of the implanted sensor 24. If in closed loop operation, then, the system 10 may optionally have one or more, preferably 2, additional pressure/temperature sensors 24. In this latter cases, one wired sensor 24 is at the brain “terminus” of the catheter 16 and the other wired sensor 24 is placed in the downstream side of the shunt valve 20 near the peritoneal end. These two sensors 24 are most preferably enclosed in a multi-lumen catheter 16 where one lumen contains the wires to operate the sensor 24 and a separate lumen is to carry the CSF fluid from the brain to the exit point in the body 40. When the sensors 24 and the μC 46 take actions, such as opening or closing the valve 20, a notice is recorded and downloaded when queried by the patient or doctor. It the valve 20 is not working as programmed or as sensed by the μC 46 then an alarm is sent to the patient and doctor.

### Third Embodiment

The third embodiment is a minimal “default” sensor system 10 with a very small footprint with an antenna 48 near the skin surface as shown in FIG. 6. It is a much simpler design and does not have an internal permanent battery 28. It has a temporary charge storage system 50, for example a capacitor, sufficiently charged by an external power source, not shown) when the external source is close enough to the system antenna 48 to send in the charge necessary to operate the system 10. The temporarily saved power is managed by the power reducing network 44 and operates the μC 46 and sensors 24 until the external patient or physician controller is removed from the proximity of the antenna 48. The system 10 does not save data but will only perform sensing when the system 10 is activated by an external power input. The sensor 24 then measures the brain pressure and sends the information to a minimal μC 46 which then reports the pressure/temperature data back to the external source such as a controller or a smart phone (which has the necessary software in an app) via a low power Bluetooth communication circuit 54. The sensor 24 may be positioned or implanted anywhere pressure/temperature measurements are needed or optionally, it may be attached or optionally incorporated into the shunt system 10. Its only function is to report back the CSF pressure when queried and powered by the external power source and controller.

Thus, it can now be appreciated that the illustrated embodiments have the following advantages. A pressure sensor is provided imbedded in a catheter tip or on ventricle external terminus. A standalone pressure sensor can be employed or attached to a shunt/catheter. A non-drifting implantable piezo sensor is employed as the pressure sensor. RFID powered non-invasive communication is provided with the shunt sensors and used to communicate pressure information to an external reader. Such a sensor enables the patient to monitor brain pressure and avoid unnecessary CT's and MRI's to say nothing about reducing patient anxiety. Patient monitoring at will is available along with remote monitoring by mobile phone connections with the attending physician.

### Optical Pressure Sensors

As diagrammatically illustrated in FIG. 8 another embodiment includes an optical pressure sensor 50 based on the distance between two walls that changes based on pressure. The sensor 50 ultimately sends an optical signal through the skin to transmit the measured pressure to an external reader 56. Thus, that optical embodiment is similar to the third embodiment described above but uses an optical sensor 50 in place of the piezo sensor 24. Since the wavelength is transparent to the skin, no internal electronics is needed. A light guide fiber 52 between the implanted CSF sensor 50 in patient 58 and a subdermal optical terminus 54 transmits light from the sensor 50 through the skin to the external reader 56.

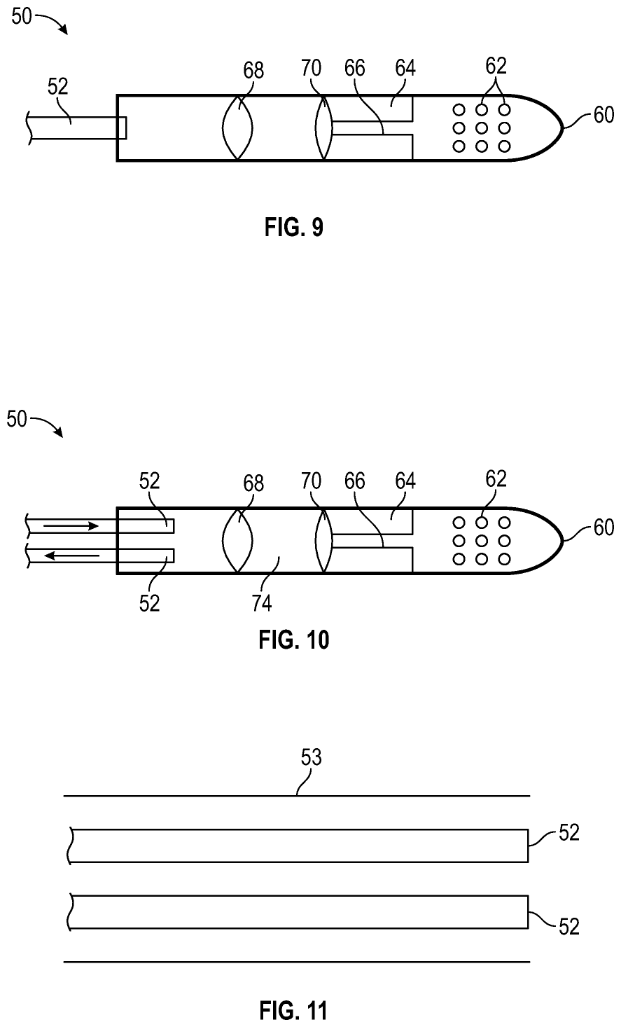

FIG. 9 is a side cross sectional view of a first embodiment for the implanted optical sensor 50, which includes a hollow bullet-shaped tip 60 having a plurality of holes 62 defined therethrough whereby CSF and pressure at the measurement site in the ventricle is communicated through a diaphragm holder 64 having a central bore 66 defined therethrough and attached to a flexible mirrored diaphragm 70, which is deflected in response to the amount of CSF pressure applied to ft, Optic fiber 52 is fitted to the end of sensor 50 and light from and to fiber 52 is focused by lens 68 onto mirrored diaphragm 70. The pressure calculation is based on the change in phase of the reflected light relative to the unreflected light of the same split light beam. The phase changes result from the changed pathlength resulting from the movement of the mirrored diaphragm 70 in the sensor 50. These changes are then determined by the reader 56 which then calculates pressure on the diaphragm 70.

The light transmitted to and from sensor 50 may be external (passive) or from an internally implanted light source electronically measured and wirelessly communicated (active). Consider first the passive optical sensor 50. Corwentionai pressure sensors applied in biomedical and biomechanical applications are based on a piezoresistive, strain gauge, or other solid-state pressure sensing technology. Light-based pressure sensing is based on a different technology. Fiber-optic pressure sensing technology is about forty years old and presents substantial advantages compared to conventional electric sensing systems. The optical pressure sensing system described herein has two main components: a fully implanted, light-based pressure sensor 50 and the reader 56. The reader 56 is a device which emits a laser light and captures the reflected light for phase comparison with the emitted light and calculates the measured pressure

The implanted pressure sensor 50 is implanted in the brain ventricle for hydrocephalus applications. A pressure sensor assembly 72 as shown in FIG. 8 comprises a pressure sensor 50, one or more optical fibers 52 that carry the light from the skin to the (brain) sensor 50 and carries the reflected light back to the skin and to an implanted optical terminus 54.

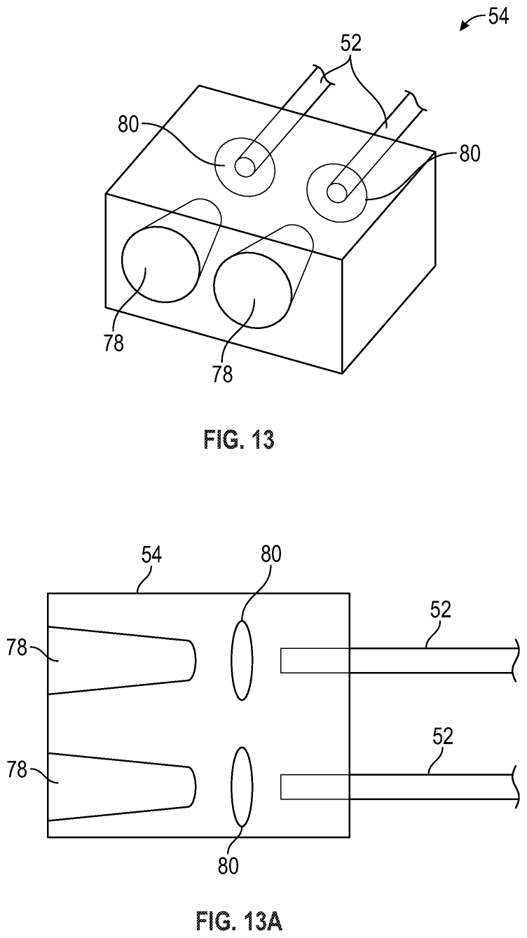

The complete assembly 72 for the example of hydrocephalus is implanted as follows. The pressure sensor 50 is inserted into the ventricle through a small burr hole cut in the skull. The fiber optic cable 52 attached to the sensor 50 is then fastened to the outside of the skull to hold it in place and so the cable 53 or sensor 50 will not move as the patient 58 moves around or turns his or her head, etc. The cable 52 is then tunneled to a skin location, where there is no significant amount of hair. For example, this could be the behind the ear, the neck or more preferably an easily accessible place on the chest in a location where it would not be unsightly or easily visible to others. The cable 52 is then connected to the subcutaneously implanted terminus 54 near enough to the surface of the skin to receive sufficient laser light intensity from the reader 56 and send reflected laser light through the skin back to the reader 5$. A transmission optic fiber 52 and a return optic fiber 52 may be provided as shown in FIGS. 13 and 13a From this location laser light from the reader 56 can pass through the skin to be sufficiently captured by the terminus 54 and ariled to the pressure sensor 50 and back to the temlinus 54 in sufficient intensity to be emitted from the terminus 54 and captured by the reader 56 that is placed close to or on the skin immediately above the terminus 54.

The pressure sensor 50 receives the light from the fiber optic cable 52. The light leaves the cable 52 and is focused by a lens 68 onto a mirrored diaphragm 70 on the opposite side of a small “air”/gas filled gap 74 between the lens 68 and the diaphragm 70. The rear side of the mirrored diaphragm 70 is open to the body fluids and, depending on the CSF pressure, will be bent inward or outward relative to the fixed internal pressure of the sensor's fixed gap 74, The bending of the diaphragm 70 thereby changes the pathiength of the light. The light is reflected by the diaphragm 70 onto lens 68 immediately before a return light fiber 52, either the same fiber if only one fiber is present or to the return fiber if two fibers are present as shown in FIG. 13. The fiber optic 52 then returns the reflected light back to the terminus 54. The tip 60 of the sensor 50 has a rounded end, sharp enough to penetrate brain tissue or a blunt end when the sensor is implanted with the use of a rioid sheath or stylet. The end 60 of the sensor 50 between the back of the mirrored diaphragm 70 and the tip 60 has a plurality of holes 62 in the side of the hollow tubing forming the wall of sensor 50 so that the CSF pressure can communicate freely with the rear side of the diaphragm 70 exposed to the CSF. This CSF fluid pressure then is able to move the diaphragm 70 in accordance with the fluidic: brain pressure. The pressure sensor 50 is biocompatible or resistant to biological fluids and is stable at body temperature. Assembly 72 operates equally well with any wavelength lioht that can be received through the skin and carried by the fiber optic cable(s) 52. FIG. 13a is a side cross sectional view of the ter riinus for dual cables 52 shown in FIG. 13 and shows the funnel-shaped optical windows 78 more clearly, FIG. 11 shows a side cross-sectional view of a dual cable included within a single sheath 53.

The fiber optic cable 52 can be made up of one or many in fiber strands as a bundle, Together this bundle 52 of individual fibers is considered as a single cable. The term cable and fiber for the purposes of this discussion are synonymous. The fiber optic cable 52 can be one or more separate cables for taking the light from the implanted optical terminus 54 to the sensor 50. If the embodiment has two different optical cables 52, then one or more cables 52 will carry the light from the tern gnus 54 to the sensor 50 and the other or separate one or more cables 52 will carry the reflected light back to the terminus 54. lf embodiment, optionally, has only a single optical cable 52, then the laser light received by the terminus 54 s carried to the pressure sensor 50. The laser light from reader 56 is timed to turn off so that it will not interfere with the reflected light being sent back to the terminus 54 on the same cable 52. In other words, in the single cable embodiment, the light pulses are modulated so the light signal is paused until the reflected light is returned and captured by the reader 56. The fiber optic cable exterior is cad in a biocompatible coating and the individual fiber's exterior below the cladding has a reflective coating 78 disposed along the fiber shank to minimize loss of light during transmission along the fiber's light path. The cable 52 and coating 76 is resistant to biological fluids and works well at body temperature, It will also be optimized to accept light from preferably the infrared and/or optionally but less preferred visible light and least preferred in the ultraviolet. Depending on the application, the light has to penetrate easily the skin to the device terminus 54 without significant loss of Intensity.

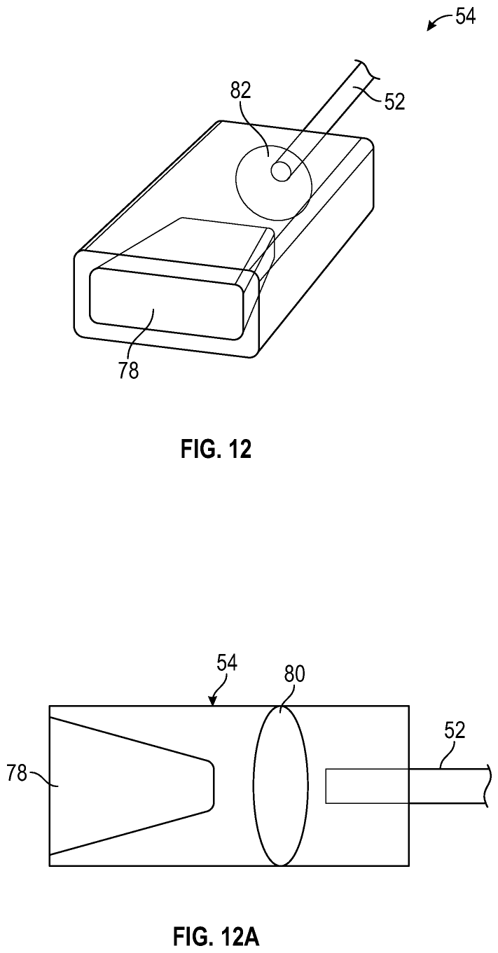

The fiber optic cable 52 terminates in a structure called the implanted optical terminus 54 implanted just below the skin. The terminus 54 has a transparent funnel-shaped guide r window 78 shown in FIG. 12 for a single cable 52 and tuned to the wavelength of the laser light coming from the reader 56 with a funnel-shaped guide 78 that focuses the light onto a lens 80 that concentrates the light and collimates it onto the fiber optic cable 52. FIG. 12a shows the terminus 54 of FIG. 12 inside cross sectional view, The terminus′ purpose is to receive pulsed light from an external source, such as reader 56, through the skin at a wavelength that is mostly transparent to the skin. The terminus 54 then focuses the it light with a lens 80 onto the optical fiber 52 which carries the incomina light to the pressure sensor 50. The reflected light from the pressure sensor 50 is then returned to the terminus 54 yvhch then emits the reflected light through the skin to the reader 56 outside the patient's body. The terminus′ exterior is resistant to biological fluids and works well at body temperature. It will also be optimized to accept light from preferably the infrared and/or optionally but less preferred visible light and least preferred in the ultraviolet. Depending on the application, the light easily penetrates the skin without significant loss of intensity. The implanted passive pressure sensing system 72 does not require any implanted electronics or battery. The system 72 has only one moving part, namely the diaphragm 70 that is moved only by CSF pressure.

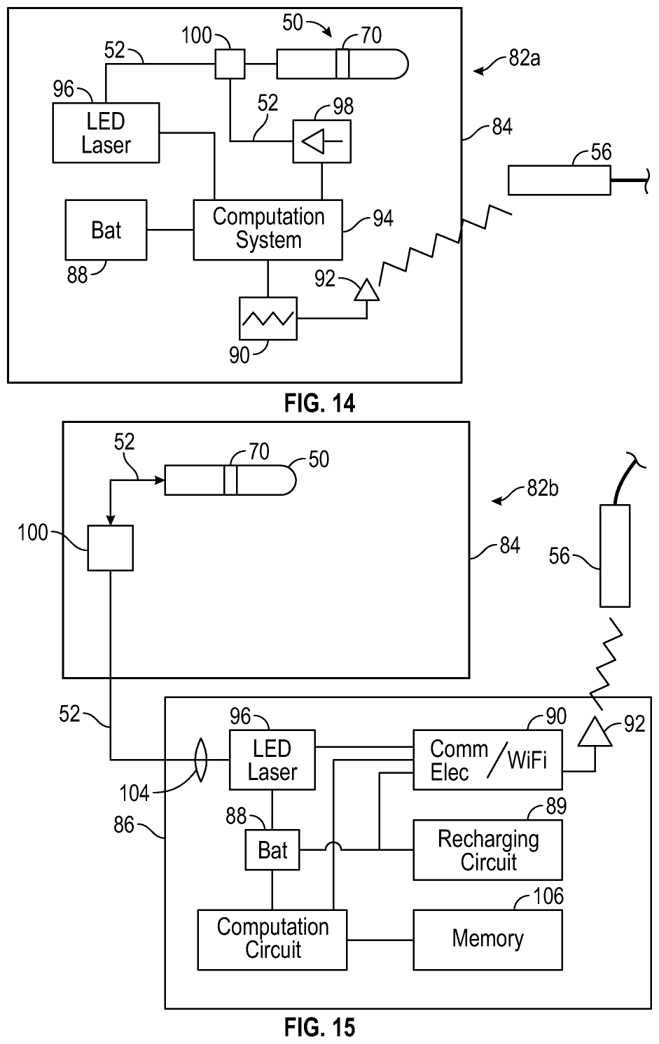

Turn now and consider the active optical sensor 50 which has a battery, electronics and laser source contained within the implanted optical pressure sensor's physical housng shown diagrammatically in FIGS. 14-17. The optical pressure sensor 82 is similar to the passive sensor 50 as it contains a diaphragm 70 that moves based on CSF pressure, There are two embodiments of the active optical sensor 82. One embodiment 82a has all the electronics and related terns viithn the body of the sensor housing 84 as shown in FIG. 14 and a second embodiment 82b that has a sensor 82b connected with a wire or optical cable 52 to an implanted electronics package 86 remote from the sensor 82b to reduce the size of the sensor 82b placed in the brain.

Consider a first embodiment of an active sensor 82a. This embodiment is an “all in one box” pressure sensing device as shown in FIG. 14. Within the sensor container body 84 with the pressure sensing assembly 72 is additionally contained a wirelessly rechargeable battery 88, Wi-Fi communication electronics 90, antenna 92, other er Ironic components to respond to reader programming, a computation system 94, a LED laser 96 to emit a laser light focused on the sensing diaphragm 70 and a light sensor 98 to receive the reflected light from the diaphragm 70. The computation system 94 processes the instructions from the reader 56 and using internal programming can send out and receive LED light, process the information and communicate it to the outside of the body to reader 56. The entire system is miniaturized to the extent possible to reduce the footprint of the device in the ventricle. The laser LED 96 also manages a control beam through a beam splitter 100 to enable it to compare the reflected beam to the original beam to reconstruct the change in distance resulting from diaphragm movement. Sensor 82a is constructed of biocompatible materials except for the diaphragm 70 The pressure sensor 82a is resistant to biological fluids and works well at body temperature. Since the light is handled entirely with n housing 84, transparency of the skin is not a consideration in the frequency choice.

Turn now to a second active sensor embodiment 82b shown in FIG. 15. Embodiment 82b has three major components: an optical pressure sensor 50, a fiber optical cable 52 that connects the sensor's optical system to a battery 88 and sensor electronics module 102 and a separate battery and electronics module 86. The fiber optic cable 52 is connected to the sensor 82b and may be one or more cables 52 depending on the design choice. The fiber optic cable(s) 52 is implanted through a burr hole in the skull and the cable 52 with sensor assembly 82b inserted into the ventricle for measuring CSF pressure, The other end of the fiber optic cable 52 is attached to the battery and electronics module 8$ outside of the skull or implanted elsewhere at a convenient location.

The battery and electronics module 86 can be attached to the exterior side of the skull near the burr hole where the fiber optic cable 52 goes through the skull. If the profile of the battery and electronics module 86 is too high for attaching to the skull below the skin, there may be a safety issue. This safety issue relates to the module 86 eroding the skin from underneath and breaking through the skin or rupturino. Depending on the level of miniaturization of the module 86 one option is to lower the profile of the device by creating a bone bed in the skull a few millimeters cut into the skull bone. Then the module 86 can be attached to the bottom of the bone bed and its profile is lowered by the necessary amount needed to not be too irritating to the skin from below. if the module's profile is too high for a bone bed, optionally, the module 86 can be placed elsewhere in the body where convenient such as subdermally on the chest. Then the fiber optic cable. 52 is tunneled from the brain where it is connected to the optical pressure sensor 82b to the battery and electronics module 86 placed elsewhere in the body. The battery and electronics module 86 contains a rechargeable battery 88, the recharging circuit 89 and software to recharge the battery through the skin, a Wi-Fi communication electronics 90 and communications software for Bluetooth communications or via Zigbee communications systems, antenna 92, computational system 94 for processing the light data and sending it to the reader 56, LED laser light 96 and lens 104 to focus the light onto the optical fiber 52, a light sensor 98 to receive the reflected light and a memory 106 to store the processed information until it is sent to the reader 56. Following transmission of data to the reader 56, the software will delete the stored but transmitted data to make room in memory 106 for the next data collection. Only the management software will be stored permanently on the module 86. This system 82b can provide near real-time brain pressure information to the reader 56. The reader 56 then takes the information and determines if an alert is needed to inform the patient and doctor of a pressure reading that indicates a need to go to the doctor or hospital emergency room or that all are in the normal pressure ranges and no action is needed by the patient and/or the doctor.

The reader 56 is a handheld device has two main embodiments. One embodiment is designed for communication with the implanted passive optical pressure sensor system 72, and the other is desioned for communlcation with the implanted active optical sensor system 82a, 82b. The reader 56 for the implanted passive optical pressure sensor system 72 must generate the laser light that will be used by the passive sensor system 72 to measure pressure, In this case, the reader 56 is put up agalnst the fiber optic terminus 54 but on the outside of the skin immediately opposite to the terminus 54 which is subcutaneous and then shines the laser light onto the terminus 54. Depending on if the passive sensor's optical fibers 52 are of a single fiber or multiple fiber design, the reader 56 either modulates its light on and off to receive the reflected light or has separate locations for the returned light sensor to receive the reflected light from the optical sensor 50. The reader 56 then processes the reflected light and compares it with the reference beam to calculate pressure change. The reader 56 then displays the pressure to the patient who is holding the reader 56 up to the terminus 54. The reader 56 additionally sends the pressure information to the doctor or healthcare professional, If the pressure is too high or too low, then it also sends a warning to the patient and the doctor of the devliation from normal CST pressure. The patient can take corrective action or go to the emergency room/or doctor to assess the problem and next steps.

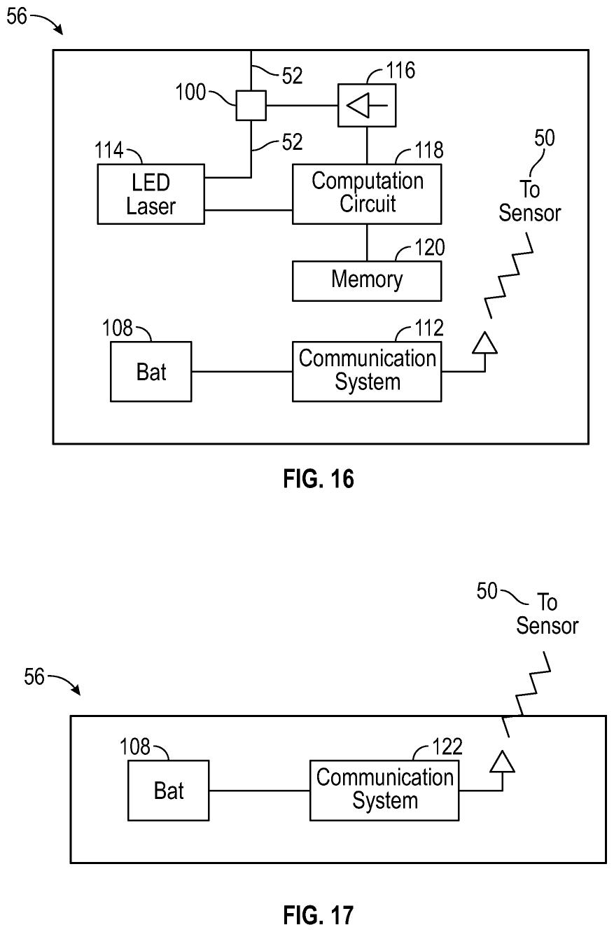

The reader 56 as shown in FIG. 16 contains a rechargeable battery 108, a wireless communication system 110 for transmitting informatlon to the healthcare professional, a wireless communication system 112 for communicating with the optical sensor 50, a LED laser 114 to query the optical sensor 50 for CSF pressure, a light sensor 116 for receiving the laser light back from the sensor 50, software driven computational electronics 118 to process the raw CSF pressure data, a memory 120 necessary to store the received processed and raw data and the programming for sending the data to a database outside of the system 72. It will also have the necessary and routine features necessary for a medical device such as onloff switches, battery power assessment, software for connecting the device to wi-fi and phone links, connectors for connection to computers for downloading data and uploading new programs,

The reader 56 for the active optical pressure sensor systems 82a and 82b is nearly identical to the reader 56 for the passive sensor system 72 except that it does not have the laser light generation and receiving function and the internal electronics for generating the light, ceiving the light and processing the information obtained by that method as shown in FIG. 17, Instead, it has the electronics 122 for receiving the electronic communications from the implanted pressure sensor 50 wirelessly and then taking that data and processing it t obtain a current CSF pressure information,

To he clear in all cases where the source of the laser light is exterior to the body or is transmitted outside of the body to render an optical measurement, the laser light frequency has to penetrate easily the skin without significant loss of intensity and without “burning” the skin from chronic and frequent use.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments include other combinations of fewer, more or different elements, which are disclosed in above even when not initially claimed in such combinations. A teaching that two elements are combined in a claimed combination is further to be understood as also allowing for a claimed combination in which the two elements are not combined with each other but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

We claim:

1. A ventricular shunt having a valve to regulate flow of cerebral spinal fluid (CSF) from the brain of a patient for treatment of hydrocephalus comprising:
a ventricular catheter for transferring CSF from a brain ventricle of a patient;
an implanted pressure and/or temperature sensor communicated with the CSF to measure pressure and/or temperature respectively of the CSF;
a wireless data transmitter to transmit the measured pressure and/or temperature to an attending physician and/or patient; and
a drainage catheter communicated to the valve for draining the CSF from the brain.

2. The shunt of claim 1 where ventricular catheter has a catheter tip, where the valve has an inlet, and where the pressure sensor is disposed at or near the ventricular catheter tip and/or at or near the inlet of the valve, so that a low pressure measurement by the pressure sensor at either the catheter tip and/or at or near the inlet of the valve less than a predetermined magnitude is reported by the wireless data transmitter as a clogged ventricular catheter to the attending physician and/or patient.

3. The shunt of claim 2 where the predetermined magnitude is 0 to 15 mmHg.

4. The shunt of claim 1 where ventricular catheter has a catheter tip, where the valve has an inlet and outlet, and where the pressure sensor includes multiple pressure sensors, selected ones of the multiple pressure sensors being disposed at or near the ventricular catheter tip and/or at or near the inlet and/or outlet of the valve, so that a high pressure measurement by selected ones of the multiple pressure sensors at or near the ventricular catheter tip and/or at or near the inlet of the valve greater than a predetermined magnitude is reported by the wireless data transmitter as a clogged valve to the attending physician and/or patient, or a high pressure measurement by selected ones of the multiple pressure sensors at or near the outlet of the valve greater than a predetermined magnitude is reported by the wireless data transmitter as a clogged drainage catheter to the attending physician and/or patient.

5. The shunt of claim 4 where the predetermined magnitude is >20 mmHg.

6. The shunt of claim 1 where a high temperature measurement by the temperature sensor greater than a predetermined magnitude is reported by the wireless data transmitter as an infection in the patient.

7. The shunt of claim 6 where the predetermined magnitude is >40° C.

8. The shunt of claim 1 where the wireless data transmitter comprises:
an external RFID scanner; and
an implantable body including:
an RFID transceiver wirelessly communicated to the RFID scanner;
a battery;
a battery charger circuit coupled to the battery and to the RFID transceiver;
a power reducing circuit; and
a programmable microcontroller coupled to the RFID transceiver, power reducing circuit and to the pressure sensor and/or temperature sensor, where the microcontroller is programmable to collect, store and/or transmit data from the pressure sensor and/or temperature sensor and to control operation of the RFID transceiver and the pressure sensor and/or temperature sensor,
whereby measured pressure and/or temperature of the CSF relating to the operational status of the shunt is wirelessly reported to a physician and/or patient for interventional response.

9. The shunt of claim 1 where the wireless data transmitter comprises:
an external RFID scanner; and
an implantable body including:
an RFID transceiver wirelessly communicated to the RFID scanner;
a battery;
a battery charger circuit coupled to the battery and to the RFID transceiver;
a power reducing circuit; and
a programmable microcontroller coupled to the RFID transceiver, power reducing circuit, to the pressure sensor and/or temperature sensor and to the valve, where the microcontroller is programmable to collect, store and/or transmit data from the pressure sensor and/or temperature sensor and to control operation of the RFID transceiver, the pressure sensor and/or temperature sensor, and the controllable valve in either a closed or open loop configuration,
whereby measured pressure and/or temperature of the CSF relating to the operational status of the shunt is wirelessly reported to a physician and/or patient when in the open loop configuration for interventional response, or is communicated to the microcontroller for control of the valve when in the closed loop configuration.

10. The shunt of claim 1 where the wireless data transmitter comprises:
an external RFID scanner; and
an implantable body including:
an antenna wirelessly communicated to the RFID scanner;
a low energy Bluetooth circuit coupled to the antenna;
a [power reducing circuit;
a capacitor for storage of power coupled to the power reducing circuit; and
a programmable microcontroller coupled to the Bluetooth circuit, power reducing circuit, and to the pressure sensor and/or temperature sensor, where the microcontroller is programmable to collect, store and/or transmit data from the pressure sensor and/or temperature sensor and to control operation of the Bluetooth circuit, the pressure sensor and/or temperature sensor,
where power is transmitted from the RFID scanner through the antenna, Bluetooth circuit, microcontroller, and power reducing circuit to the capacitor,
whereby measured pressure and/or temperature of the CSF relating to the operational status of the shunt is wirelessly reported to a physician and/or patient for interventional response.

11. A method of using a ventricular shunt having a controllable valve to regulate flow of cerebral spinal fluid (CSF) from the brain of a patient for treatment of hydrocephalus comprising:
delivering CSF from the ventricle of a brain of a patient through a ventricular catheter;
measuring pressure and/or temperature of the CSF using a pressure sensor and/or temperature sensor respectively communicated to the ventricular catheter as delivered to the pressure and/or temperature sensor;
transmitting the measured pressure and/or temperature to a physician and/or patient using a wireless data transmitter;
regulating flow of the CSF using a controllable valve communicated to the pressure sensor and/or temperature sensor; and
transferring the CSF through a drainage catheter communicated to the controllable valve to a body situs for CSF absorption.

12. The method of claim 11 where measuring pressure of the CSF determines if a low-pressure measurement by the pressure sensor is less than a predetermined magnitude and reporting the determined low pressure using the wireless data transmitter to the attending physician and/or patient as a clogged ventricular catheter.

13. The method of claim 12 where the predetermine magnitude is <0 mmHg.

14. The shunt of claim 11 where measuring pressure of the CSF determines if a high-pressure measurement by the pressure sensor is greater than a predetermined magnitude and reporting the determined high pressure to the attending physician using the wireless data transmitter to the attending physician as a clogged programmable valve or clogged peritoneal tubing.

15. The shunt of claim 14 where the predetermined magnitude is >20 mmHg.

16. The method of claim 11 where measuring the temperature determines if a high temperature measurement by the temperature sensor is greater than a predetermined magnitude and reporting the high temperature to the physician and/or patient using the wireless data transmitter as an infection in the patient.

17. The method of claim 16 where the predetermined magnitude is >40° C.

18. The method of claim 11 where regulating flow of the CSF using a controllable valve communicated to the pressure sensor and/or temperature sensor is performed by physician and/or patient intervention in an open loop control of the valve.

19. The method of claim 11 where regulating flow of the CSF using a controllable valve communicated to the pressure sensor and/or temperature sensor is controlled by a programmable microcontroller coupled to the valve and to the pressure and/or temperature sensor for closed loop control of the valve.

20. The method of claim 11 where the ventricular catheter has a catheter tip, and the valve has an inlet and outlet, where the pressure and/or temperature sensor include a plurality of pressure sensors communicated to the CSF at or near the catheter tip, the inlet or the outlet of the valve, where measuring pressure and/or temperature of the CSF is performed at or near the catheter tip, the inlet or the outlet of the valve, and where regulating flow of the CSF using a controllable valve communicated to the pressure sensor and/or temperature sensor is performed based on an interpreted operational status of the shunt based on pressure and/or temperature measurements at or near the catheter tip, and/or the inlet or the outlet of the valve.

21. An optical sensor system for measuring CSF pressure in a brain of a patient comprising:
an implanted optical sensor for receiving light and returning phase shifted light indicative of the CSF pressure in the brain;
source of light for generating light communicated to the optical sensor;
an exterior reader for reading the CSF pressure in the brain as indicated in the returned phase shifted light from the optical sensor; and
means for communicating the CSF pressure in the brain as indicated in the returned phase shifted light from the optical sensor to the reader or for communicating the returned phase shifted light from the optical sensor to the reader.

22. The optical sensor system of claim 21 where the source of light is exterior to the patient and where the means for communicating the returned phase shifted light from the optical sensor to the reader comprises an implanted optical terminus and an optical cable communicating the implanted optical sensor with the implanted terminus, the exterior reader optically coupling with the implanted terminus.

23. The optical sensor system of claim 21 where the source of light is implanted in the patient and where the means for communicating the CSF pressure in the brain as indicated in the returned phase shifted light from the optical sensor to the reader comprises an implanted wireless communication circuit electromagnetically communicating with the exterior reader.

24. The optical system of claim 23 where the source of light and means for communicating the CSF pressure are included within a single implanted housing.

25. The optical system of claim 23 where the source of light and means for communicating the CSF pressure are included within a plurality of intercommunicated implanted housings.
